(19) United States
(12) Patent Application Publication
(10) Pub. No.: US 2010/0114120 A1
(43) Pub. Date: May 6, 2010

(54) Assemblies for deploying fasteners in tissue and snares for use in such assemblies

(76) Inventors: Steve G. Baker, Redmond, WA (US); Scott Harshman, Kirkland, WA (US); Hanh To, Bellevue, WA (US); Sean Totten, Kirkland, WA (US)

Correspondence Address: HOEKENDIJK & LYNCH, LLP, P.O. BOX 4787, BURLINGAME, CA, 94011-4787, US

(21) Appl. No.: 12/655,645

(22) Filed: Jan. 5, 2010

Related U.S. Application Data
(63) Continuation of application No. 11/543,720, filed on Oct. 4, 2006.

(51) Int. Cl. A61B 17/10

(52) U.S. Cl. 606/139

(57) ABSTRACT

An assembly is provided to facilitate the deployment of at least one fastener through tissue, such as stomach tissue. The assembly comprises a fastener deploying device arranged to deploy a fastener through the tissue and a snare arranged to bind the tissue and fastener deploying device together as the fastener deploying device deploys a fastener through the tissue.

### FIELD OF THE INVENTION

The present invention generally relates to assemblies for deploying fasteners in tissue. The present invention more particularly relates to such assemblies for deploying tissue fixation devices wherein the assemblies include at least one snare for stabilizing the tissue during fastener deployment.

### BACKGROUND

Tissue fixation devices or fasteners find many different uses in the medical field. For example, in surgery, multiple tissue layers are often fixed together to maintain the tissue in a desired shape or configuration, or to effect a desired seal with the tissue. Unfortunately, tissue fixation is not easily accomplished in the human body. This results because the tissue to be fastened often does not lend itself to stabilization. The tissue can be too slippery or lack structural integrity or texture to obtain a good hold on the tissue to enable fastener placement where desired or required. Tissue stabilization is thus important in tissue fixation to assure that after fastener deployment, the tissue does indeed have the desired shape or provides the desired seal.

Fastener deployment is also complicated by limited space in which to localize the tissue or accommodate fastener deployment tools. For example, tissue stabilization for fastening is particularly difficult when the fastening devices must be passed through a patient's mouth and down the esophagus. Further, visualization of the fastener deployment site can also be limited by both lack of light and interfering tissue.

Hence, there is a need in the art for improved tissue fastener deployment assemblies. More particularly, there is a need in the art for such assemblies, which may be readily placed in the body, and which afford improved tissue stabilization notwithstanding varying tissue structures, textures, and accessibilities.

### SUMMARY

The invention provides an assembly for deploying a fastener through tissue. The assembly comprises a fastener deploying device arranged to deploy a fastener through tissue and a snare arranged to bind the tissue and fastener deploying device together as the fastener deploying device deploys a fastener through the tissue.

The snare may include a cable loop arranged to encircle the tissue and fastener deploying device to bind the tissue and fastener deploying device together. The snare may further include an elongated member having a proximal end, a distal end, and at least one longitudinal lumen extending between the proximal end and the distal end, and the cable loop may extend from the distal end of the elongated member through the at least one longitudinal lumen.

The elongated member may be retroflexed at the distal end. The fastener deploying device may include a passageway and the elongated member may extend through the passageway. The elongated member may be an endoscope.

The assembly may further comprise a mechanical retractor that pulls the tissue through the snare. The mechanical retractor may include a helical coil that grabs the tissue.

The snare may include a cable formed in a loop including first and second ends and a spreader that increases the spacing between the ends of the loop. The snare spreader may comprise a spreader block having a pair of lumens. The lumens may terminate in respective spaced apart openings defining the spacing between the cable loop ends. The spreader block may include a tissue engaging surface between the spaced apart openings against which the tissue layers lie. The snare may further comprise an opposing block carried on the cable having an opposing tissue engaging surface so that the tissue engaging surface and the opposing tissue engaging surface may confine the tissue there between. The spreader block and the opposing block may each be hollowed out. The opposing block may be hollowed out to provide, for example, clearance for a deployed fastener to pass through the tissue.

The snare may include a plurality of cable loops arranged to encircle the tissue and fastener deploying device to bind the tissue and fastener deploying device together. The plurality of cable loops may be arranged in side-by-side relation. The plurality of cable loops may be a pair of cable loops arranged in side-by-side relation. The fastener deploying device may then be arranged to deploy a fastener between the pair of cable loops.

In another embodiment, the invention provides an assembly for deploying a fastener through tissue, comprising a snare including an elongated member having a proximal end, a distal end, and at least one longitudinal lumen extending between the proximal end and the distal end, and a cable loop extending from the distal end of the elongated member through the at least one longitudinal lumen. The assembly further comprises a fastener deploying device arranged to deploy a fastener. The cable loop of the snare is arranged to encircle the tissue and fastener deploying device and bind the tissue and fastener deploying device together as the fastener deploying device deploys a fastener through the tissue.

In another embodiment, the invention provides a method of deploying a fastener through tissue. The method comprises the steps of providing a fastener deploying device arranged to deploy a fastener through tissue, providing a snare arranged to bind the tissue and fastener deploying device together as the fastener deploying device deploys a fastener through the tissue, binding the tissue and the fastener deploying device together with the snare, and deploying a fastener through the tissue with the fastener deploying device.

The snare may include a cable loop and the binding step may include the step of encircling the tissue and fastener deploying device with the cable loop to bind the tissue and fastener deploying device together. The snare may further includes an elongated member having a proximal end, a distal end, and at least one longitudinal lumen extending between the proximal end and the distal end with the cable loop extending from the distal end of the elongated member through the at least one longitudinal lumen, and the binding step may further include retroflexing the distal end of the elongated member. The fastener deploying device may include a passageway and the method may further comprise the step of feeding the elongated member down and through the passageway. The elongated member may be an endoscope and the method may further comprise the step of viewing the tissue and fastener deploying device through the endoscope with the distal end of the endoscope retroflexed.

The method may further comprise the step of pulling the tissue through the snare. The pulling step may include grabbing the tissue with a helical coil.

The snare may include a cable formed in a loop including first and second ends and the method may further comprise the step of spreading the ends of the loop. The deploying step may then include directing a fastener through the tissue between the spread ends of the cable loop. The snare may include a pair of cable loops and the deploying step may include driving the fastener through the tissue between the pair of cable loops.

### BRIEF DESCRIPTION OF THE DRAWINGS

The features of the present invention which are believed to be novel are set forth with particularity in the appended claims. The invention, together with further objects and advantages thereof, may best be understood by making reference to the following description taken in conjunction with the accompanying drawings, in the several figures of which like reference numerals identify like elements, and wherein:

FIG. 1 is a perspective side view with portions cut away of a stomach and a fastener deployment assembly including a fastener deployment device and a snare according to a first embodiment of the invention in use to deploy at least one fastener to maintain a gastroesophageal valve (GEV) therein;

FIG. 2 is a perspective side view with portions cut away of the stomach and the fastener deployment assembly of FIG. 1 during an initial stage of its use;

FIG. 3 is a perspective side view with portions cut away of the stomach and the fastener deployment assembly of FIG. 1 during a further stage of its use to deploy a fastener;

FIG. 4 is a perspective side view with portions cut away of the stomach and the fastener deployment assembly of FIG. 1 as the snare captures the fastener deployment device during a further stage of its use to deploy a fastener;

FIG. 5 is a perspective side view with portions cut away of the stomach and the fastener deployment assembly of FIG. 1 during a further stage of its use and as a fastener is being delivered for deployment;

FIG. 6 is a perspective side view with portions cut away of the stomach and the fastener deployment assembly of FIG. 1 showing the fastener deployment device being captured during an initial stage of an alternative method of capture;

FIG. 7 is a perspective side view with portions cut away of the stomach and the fastener deployment assembly of FIG. 1 showing the fastener deployment device being captured according to the alternative method of capture;

FIG. 8 is a side plan view of a fastener deployment assembly according to a further embodiment of the invention;

FIG. 9 is a perspective side view with portions cut away of a stomach and the fastener deployment assembly of FIG. 8 during an initial stage of its use;

FIG. 10 is a perspective side view with portions cut away of the fastener deployment assembly of FIG. 8 during a further stage of its use and as a fastener is being deployed;

FIG. 11 is a perspective side view with portions cut away of the fastener deployment assembly of FIG. 8 after the fastener has been deployed;

FIG. 12 is a perspective side view with portions cut away of a further fastener deployment assembly according to another embodiment of the invention;

FIG. 13 is a perspective side view, with portions cut away, of still another fastener deployment assembly embodying the invention; and

FIG. 14 is a perspective side view showing how the fastener deployment assembly may be employed to deploy a fastener.

### DETAILED DESCRIPTION

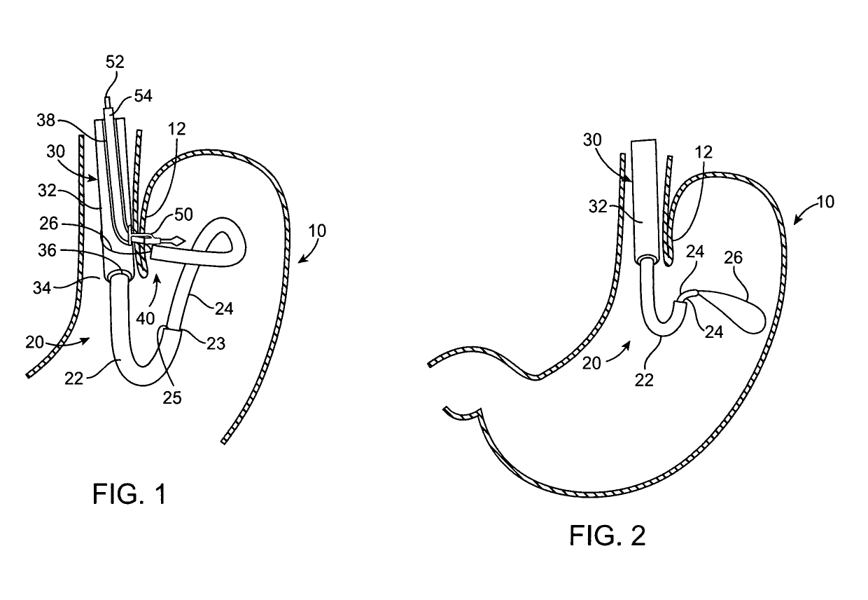

FIG. 1 is a perspective side view with portions cut away of a stomach 10 and a fastener deployment assembly 30 embodying the present invention. The assembly generally includes a fastener deployment device 30 and a snare 20 shown in use to deploy at least one fastener for maintaining a gastroesophageal valve 12.

The fastener deployment device 30 includes an elongated body 32. The body 32 has a distal end 34 extending into the stomach 10. The body includes a through channel 36 that sliding receives the snare 20. The body further has a fastener-directing channel 38 that directs a fastener 50 into the stomach tissue to be fastened.

The snare 20 includes an elongated member 22, a catheter 24, and a cable 40 that forms a cable loop 26. The elongated member is preferably an endoscope having a distal end 23, a proximal end (not shown) and a lumen 25 that receives the catheter 24. The endoscope 22 may thus be used for both introducing the snare into the stomach and providing visualization of the fastener deployment process.

The fastener 50 may take the form of the fasteners and be deployed as shown and described, for example, in co-pending application Ser. No. 11/043,903, filed Jan. 25, 2005, for SLITTED TISSUE FIXATION DEVICES AND ASSEMBLIES FOR DEPLOYING THE SAME, which application is incorporated herein in its entirety. To that end, the fastener 50 is carried on a deployment wire or stylet 52 and the leading member thereof is pushed by a pusher 54 into and through the tissue. In accordance with aspects of the invention, during the fastener deployment process, and as illustrated in FIG. 1, the fastener deployment device 30 and the tissue of valve 12 are bound together by the snare 20. This provides stabilization of the tissue to permit the fastener 50 to be accurately placed and forced through the tissue. FIGS. 2-5 illustrate a manner in which the fastener deployment of FIG. 1 may be achieved.

In FIG. 2 it may be seen that the endoscope 22 has been passed down the elongated member 32 and retroflexed to provide visualization of the process. Thereafter, the catheter 24 is pushed out of the distal end of the endoscope 22. The cable loop 26 is then pushed out of the distal end of the catheter 24.

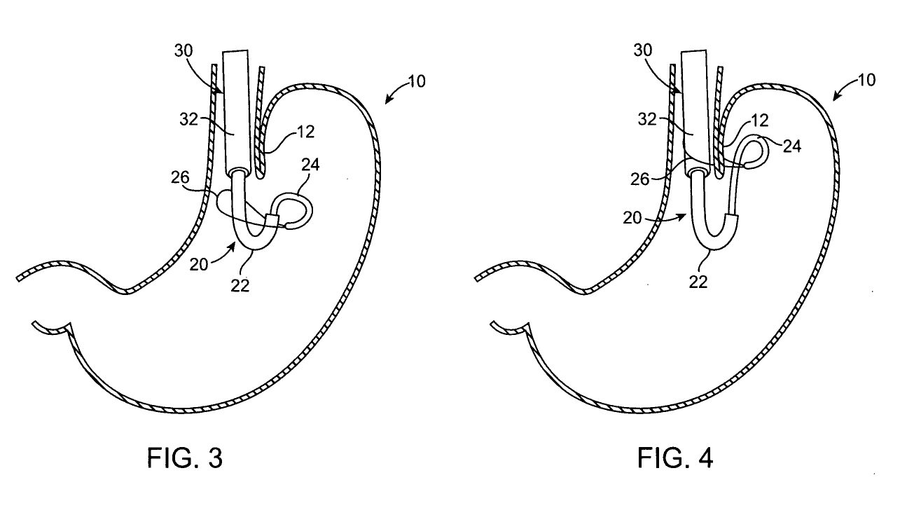

The catheter 24 preferably has shape-memory so that as the catheter 24 is advanced further distally, it will assume the shape illustrated in FIG. 3. More specifically, as the catheter 24 is played out, it curls around to cause the cable loop 26 to be loosely received over the endoscope 22.

Referring now to FIG. 4, with the cable loop 26 loosely received on the endoscope 22, the whole assembly may now be translated in an oral or proximal direction. The assembly is translated enough to position the cable loop 26 around both the fastener deployment device 30 and the tissue 12. The assembly is now ready to bind the fastener deployment device 30 and the tissue 12 together.

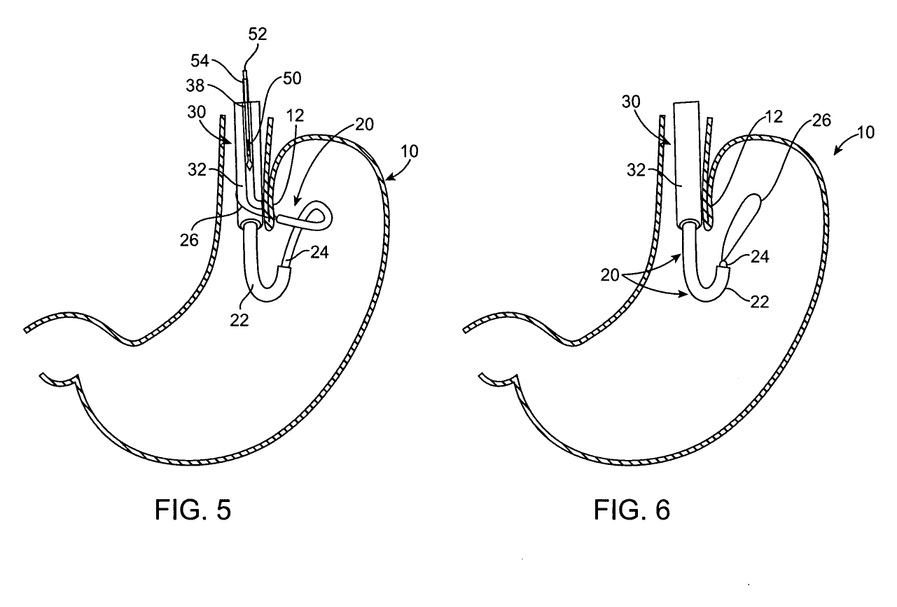

As will be noted in FIG. 5, the cable loop is now pulled tight to bind the fastener deployment device 30 to the tissue 12. A fastener 50 may now be pushed down the stylet 52 by the pusher 54 within the channel 38 to the tissue as shown in FIG. 1 for deployment.

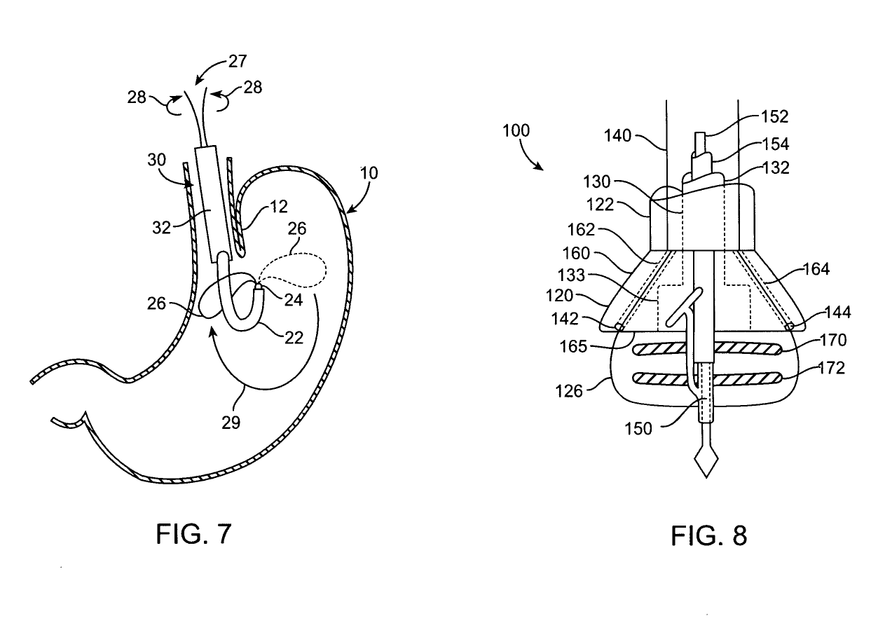

FIGS. 6 and 7 show another way in which the cable loop 26 may be manipulated to loosely receive the fastener deployment device 30 and the tissue 12. Once again the endoscope 22 is retroflexed to permit visualization. Instead of plying out the catheter 24, the proximal ends 27 of the cable loop 26 are twisted around as indicated by the arrows 28. This will cause the loop 26, which is preferably preformed in a bent structure, to swing around as shown in FIG. 7 from the dashed line position to the solid line position. The cable loop 26 is now loosely received over the fastener deployment device 30 and the tissue 12. The assembly is now ready to deploy a fastener as shown in FIGS. 1 and 5.

Referring now to FIG. 8, it shows another embodiment of the present invention. The assembly 100 of FIG. 8 generally includes a snare 120 and a fastener deployment device 130.

The snare 120 includes an elongated conduit 122 that guides two legs of a cable 140 that form a loop 126. The snare also includes a spreader block 160. The spreader block 160 has diverging lumens 162 and 164 that terminate at a spreader surface 165. The diverging lumens 162 and 164 increase the spacing between the ends 142 and 144 of the cable loop 126.

The fastener deployment device 130 includes a conduit 132 that extends down the conduit 122. The conduit 132 terminates with an enlarged section 133 at the spreader surface 165.

As will be seen subsequently, tissue to be fastened is pulled through the cable loop 126. The tissue engaging the spreader surface 165 is caused to spread to form tissue layers 170 and 172. The cable 140 may then be drawn tight causing the cable loop 126 to securely hold the tissue layers against the spreader surface 165. Now, the stylet 152 may be guided by the conduit 132 into and through the tissue layers 170 and 172. The fastener 150 is then pushed down the stylet 152 by the pusher 154 into the tissue layers 170 and 172. The enlarged portion 133 of the conduit 132 is provided to accommodate the fastener 150 and to provide space for its deployment.

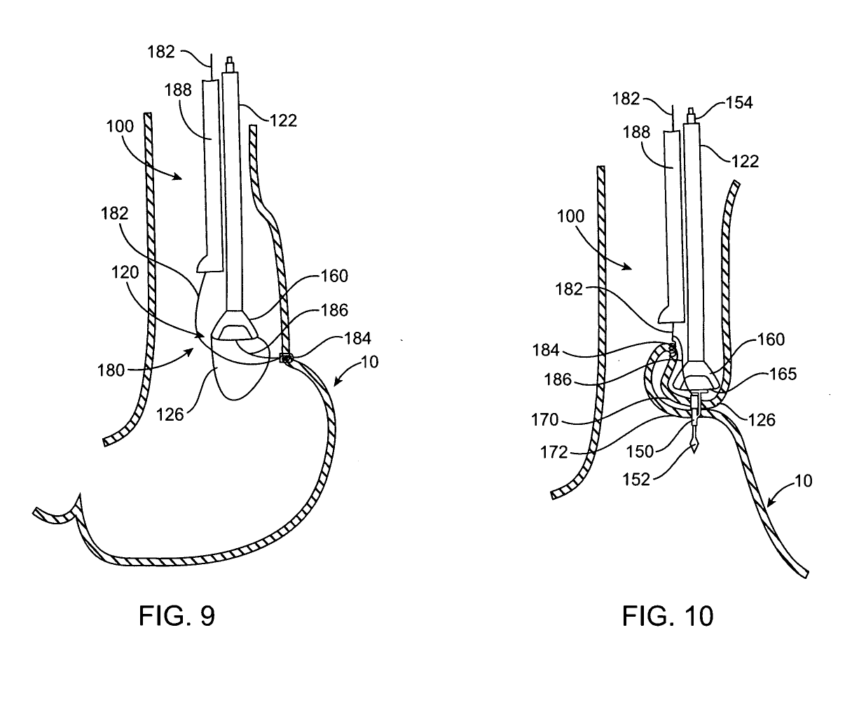

Referring now to FIGS. 9 through 11, they show a manner in which the assembly 100 of FIG. 8 may be employed to fasten tissue layers of a stomach 10. In FIG. 9, it will be noted that the conduit 122 has been fed into the stomach 10. The assembly 100 also includes a mechanical retractor 180 comprising another conduit or catheter 188, a first cable 182, and a second cable 186. The first cable 182 extends through the cable loop 126 and terminates in a tissue grabber, such as a helical coil 184 that, when engaged with and rotated against tissue, grabs the tissue. Accordingly, as seen in FIG. 9, the helical coil 184 has been rotated against the tissue of the stomach 10 and has grabbed the tissue. As may be appreciated, other forms of tissue grabbers may be employed in place of the helical coil 184. The second cable 186 extends down the conduit 122. It serves to provide guiding assistance to the first cable 182 when the helical coil is directed to the tissue.

The cable 182 is now used to pull the tissue through the cable loop 126. As may be seen in FIG. 10, the tissue is pulled through the cable loop 126 and along side the spreader block and the conduit 122. The spreader surface 165 forms a snare base that exerts a force against the tissue. Here, that force is in the direction of travel by the fastener 150. The tissue is pulled by the cable 182 at an angle and in a direction having a component opposed to the direction of the force applied to the tissue by the snare base. In the process, the tissue layers 170 and 172 are formed against the spreader surface 165. The cable loop 126 may now be drawn tight and the fastener 150 may be deployed as previously described.

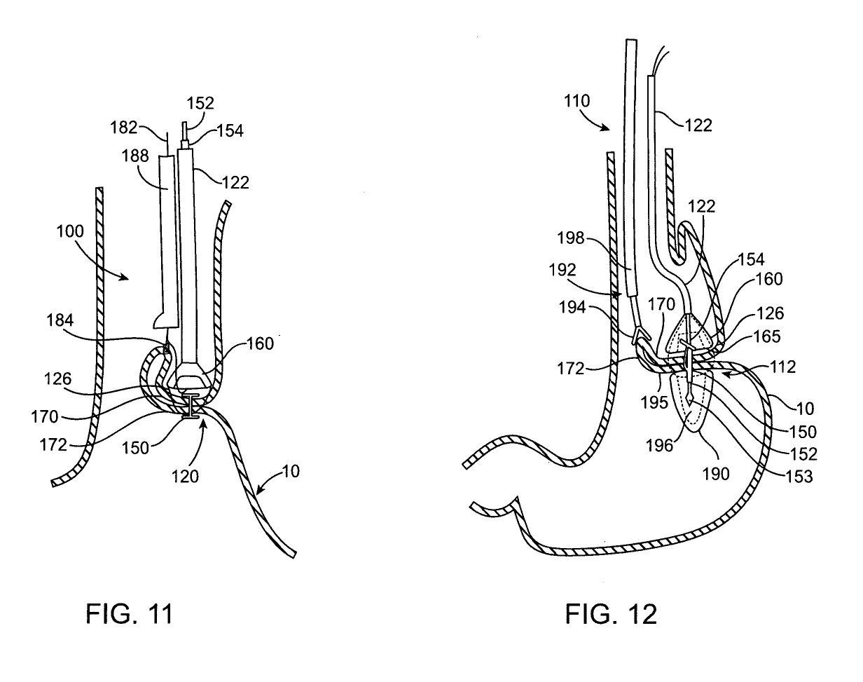

FIG. 11 shows the fastener 150 deployed through the tissue layers 170 and 172. The cable loop 126 has been loosened to permit the fastened tissue to be removed from the snare after the helical coil 184 is counter-rotated out of the tissue.

FIG. 12 shows another embodiment of the invention. The assembly 110 there shown is substantially similar to the assembly 100 except for the fact that the snare 112 of the assembly 110 includes an opposing block 190 that opposes spreader block 160 and that the assembly 110 employs an alternate form of mechanical tissue retractor 192. Firstly, with respect to the mechanical retractor 192, it will be seen that it includes a tissue forceps 194 instead of a helical coil for grabbing the tissue. With respect to the opposing block 190, it provides an opposing surface 195 juxtaposed to the surface 165 of the spreader block 160 to confine the tissue to be fastened there between. The opposing block 190 has a hollowed out portion 196 that receives the tip 153 of the stylet 152 to protect surrounding tissue from the tip 153 and to provide space for the fastener 150. In use, the operation of the assembly 110 is substantially the same as that described with respect to the assembly 100 of FIGS. 9 through 11.

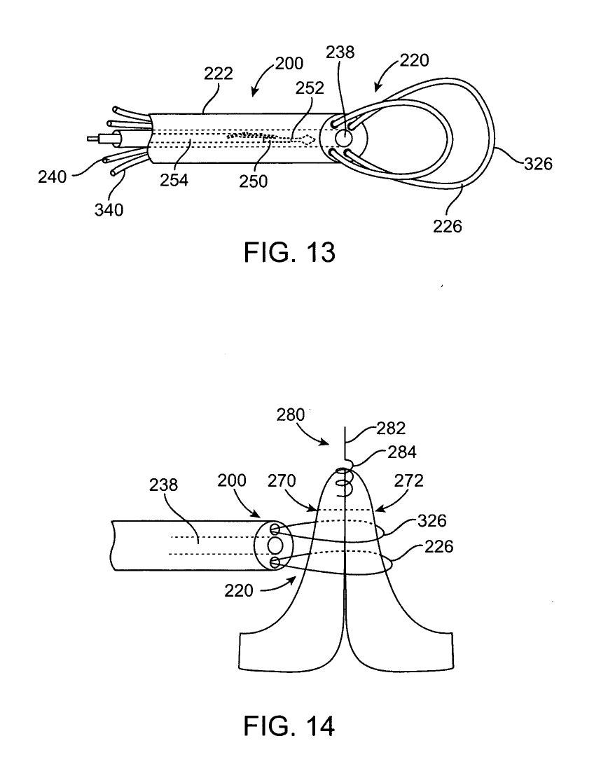

Referring now to FIGS. 13 and 14, they illustrate another assembly 200 embodying the invention. Here, the assembly includes a snare 220 comprising a pair of cable loops 226 and 326 formed from cables 240 and 340 respectively. The legs of the cables 240 and 340 extend down a conduit 222 includes a channel 238 to accommodate the fastener deployments stylet 252 and pusher 254 to enable deployment of fastener 250. The channel extends down the conduit 222 to place the fastener in between the cables loops 226 and 326.

FIG. 13 schematically shows the assembly 200 in use. Here it may be seen that a mechanical retractor 280 includes a cable 282 and helical coil 284. They have been employed to pull the tissue layers 270 and 272 through both cable loops 226 and 326. The two spaced cable loops 226 and 326 function together to prevent the tissue form slipping out of the grip of the snare 220. The snare 220 thus holds the tissue against the channel to permit a fastener to be deployed by the stylet 252 and pusher 254 as previously described.

While the invention has been described by means of specific embodiments and applications thereof, it is understood that numerous modifications and variations may be made thereto by those skilled in the art without departing from the spirit and scope of the invention. It is therefore to be understood that within the scope of the claims, the invention may be practiced otherwise than as specifically described herein.

1-61. (canceled)

62. A method of fastening stomach tissue together, comprising the steps of:
providing a tissue fastening assembly including a cable loop, a tissue retractor and a fastener deployment device having a fastener;
delivering the tissue fastening assembly down a throat of a patient to the patient's stomach;
grabbing stomach tissue with the tissue retractor;
pulling the stomach tissue into the cable loop;
drawing the cable loop around the stomach tissue after the pulling step;
applying a fastener through the stomach tissue after the drawing step; and
opening the cable loop to release the stomach tissue after the applying step.

63. The method of claim 6, wherein:
the providing step is carried out with the tissue fastening assembly including a tissue engaging surface positioned between opposing sides of the cable loop;
the pulling step being carried out to pull the stomach tissue under tension against the tissue engaging surface.

64. The method of claim 63, wherein:
the pulling step is carried out with the stomach tissue being pulled proximally to apply tension to the stomach tissue.
